(19) United States
(12) Patent Application Publication
(10) Pub. No.: US 2021/0222066 A1
(43) Pub. Date: Jul. 22, 2021

(54) LIQUID CRYSTAL COMPOUND, LIQUID CRYSTAL COMPOSITION AND PHOTOELECTRIC DISPLAY DEVICE THEREOF

(71) Applicant: Jiangsu Hecheng Display Technology Co., Ltd., Yangzhong (CN)

(72) Inventors: Wenming Han, Yangzhong (CN); Haibin Xu, Yangzhong (CN); Wenqi Zhang, Yangzhong (CN); Di He, Yangzhong (CN)

(73) Assignee: Jiangsu Hecheng Display Technology Co., Ltd., Yangzhong (CN)

(21) Appl. No.: 16/755,204

(22) PCT Filed: Dec. 11, 2018
(86) PCT No.: PCT/CN2018/120225

§ 371 Date: Apr. 10, 2020

(30) Foreign Application Priority Data
Dec. 21, 2017 (CN) 201711395076.9

(51) Int. Cl. C09K 19/34; C07D 319/06

(52) U.S. Cl. CPC C09K 19/3402; C09K 2019/3422; C07D 319/06

(57) ABSTRACT

A liquid crystal composition includes the compound of general formula I, and a photoelectric display device also includes the liquid crystal composition of general formula I. The compound of general formula I enables the liquid crystal composition including the same to maintain larger dielectric anisotropy, have a desired level of optical anisotropy, higher photo-thermal stability and nematic phase stability, and have a wide range of applicabilities, especially applicable to the IPS-type and TN-TFT-type liquid crystal display devices.

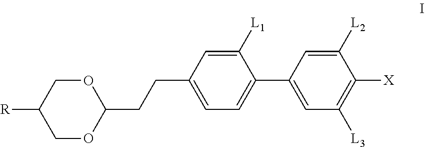

### CROSS-REFERENCE TO RELATED APPLICATIONS

This application is the National Stage of International Application No. PCT/CN2018/120225, filed Dec. 11, 2018, which claims the benefit of Chinese Application No. 201711395076.9, filed Dec. 21, 2017, the contents of which is incorporated by reference herein.

### TECHNICAL FIELD

The present invention relates to the field of liquid crystal material, particularly to a liquid crystal compound and liquid crystal composition as well as photoelectric display device thereof.

### BACKGROUND ARTS

Liquid crystal display elements using a liquid crystal composition are widely used in displays such as clocks, calculators, word processors, and the like. These liquid crystal display elements take advantage of optical anisotropy, dielectric anisotropy, and the like of liquid crystal compound. The known operating modes of liquid crystal display elements are mainly classified into the types of PC (phase change), TN (twist nematic), STN (super twisted nematic), ECB (electrically controlled birefringence), OCB (optically compensated bend), IPS (in-plane switching), VA (vertical alignment), and the like. In recent years, studies on applying an electric field to an optically isotropic liquid crystal phase to exhibit an electric birefringence mode are also prevalent.

Based on the driving mode of devices, they are mainly classified into passive matrix (PM), which is classified into the types of static, the multiplex and the like, and active matrix (AM), which is classified into the types of thin film transistor (TFT), metal insulator metal (MIM) and the like.

These liquid crystal display elements comprise liquid crystal compositions having appropriate physical properties. The general physical properties necessary for a liquid crystal compound which is used as a component of a liquid crystal composition are as follows:

(1) chemical stability and physical stability;

(2) high clearing point (liquid crystal phase-isotropy phases transition temperature);

(3) low minimum temperature of the liquid crystal phase (e.g., an optically isotropic liquid crystal phase, such as a nematic phase, a cholesterol phase, a smectic phase and a blue phase, and the like);

(4) excellent compatibility with other liquid crystal compounds;

(5) appropriate dielectric anisotropy;

(6) appropriate optical anisotropy.

Low-voltage driving is required for TFT-type liquid crystal display devices. In order to meet this requirement, it is necessary that the liquid crystal compound and the liquid crystal composition have higher Δε. Thus, a liquid crystal material having high voltage holding ratio VHR and high Δε is actively developed. Chinese patent application CN104837955A discloses a liquid crystal composition comprising a compound with the following structure:

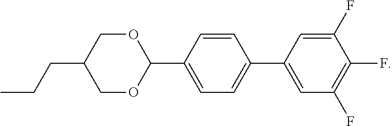 [SMILES: CCCC1COC(c2ccc(-c3cc(F)c(F)c(F)c3)cc2)OC1]

Although this liquid crystal compound has the characteristics of high Δε, it has problems of poor compatibility with other liquid crystal components and easy crystallization when stored for a long time.

### SUMMARY OF THE INVENTION

Objects: In view of the defects in the prior art, the object of the present invention is to provide a liquid crystal compound, which enables the liquid crystal composition comprising the same to maintain larger dielectric anisotropy, have a desired level of optical anisotropy, higher photo-thermal stability and nematic phase stability, and have a wide range of applicabilities, especially applicable to the IPS-type and TN-TFT-type liquid crystal display devices.

Another object of the present invention is to provide a liquid crystal composition comprising the liquid crystal compound and a photoelectric display device comprising the liquid crystal composition.

### TECHNICAL SOLUTIONS OF THE PRESENT INVENTION

In one aspect, the present invention provides a compound having the structure of general formula I:

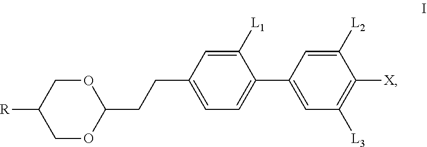

in which,

R represents a C₁₋₁₂ linear or branched alkyl or alkoxy, a C₂₋₁₂ linear or branched alkenyl or alkenoxy, or a C₃₋₁₂ cycloalkyl, wherein one or more —CH₂— in the alkyl or alkoxy, alkenyl or alkenoxy, or cycloalkyl can be replaced by —O—, provided that oxygen atoms are not directly connected;

L₁, L₂ and L₃ each independently represents —H or —F;

X represent —F, a C₁₋₁₂ linear or branched fluorinated alkyl or fluorinated alkoxy, or a C₂₋₁₂ linear or branched fluorinated alkenyl or fluorinated alkenoxy.

In some embodiments of the present invention, X preferably represents —F, —CF₃ or —OCF₃.

In some embodiments of the present invention, R further preferably represents a C₁₋₇ chain alkyl or chain alkoxy, or a C₂₋₁₂ chain alkenyl.

The preferred chain alkyl is, for example, methyl, ethyl, n-propyl, isopropyl, n-butyl, isobutyl, sec-butyl, t-butyl, 2-methylbutyl, n-pentyl, sec-pentyl, and the like.

The preferred chain alkoxy is, for example, methoxy, ethoxy, 2-methoxyethoxy, n-propoxy, isopropoxy, n-butoxy, isobutoxy, sec-butoxy, t-butoxy, 2-methylbutoxy, n-pentyloxy, and the like.

The preferred chain alkenyl is, for example, ethenyl, propenyl, butenyl and pentenyl.

In some embodiments of the present invention, the compound of general formula I is preferably selected from a group consisting of the following compounds:

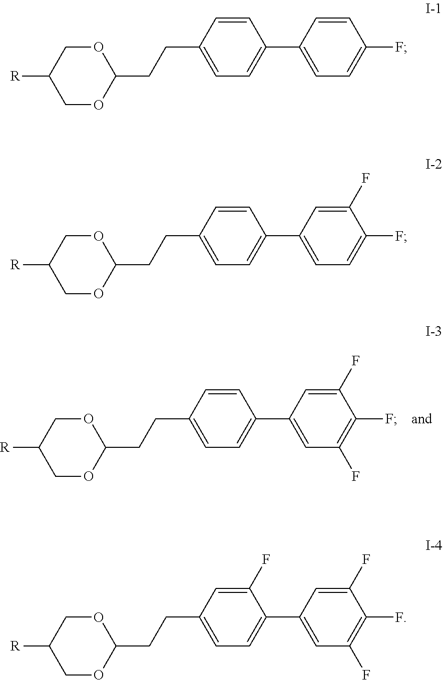

In some embodiments of the present invention, the compound of general formula I-1 is further preferably selected from a group consisting of the following compounds:

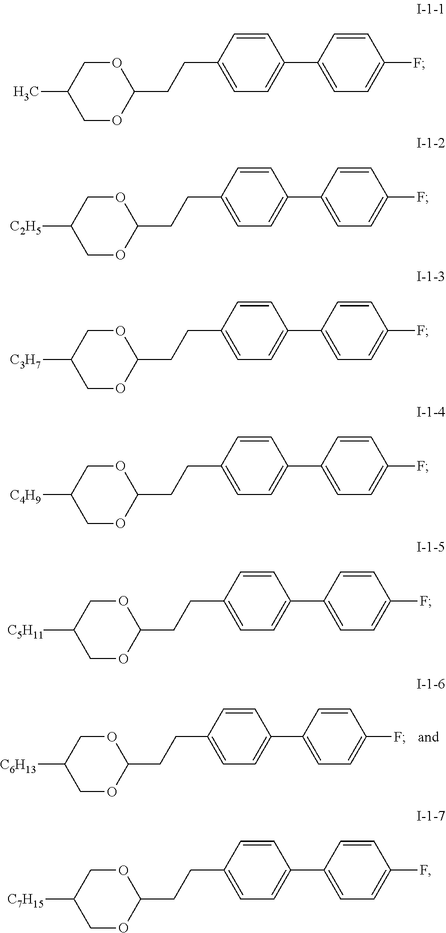

the compound of general formula I-2 is selected from a group consisting of the following compounds:

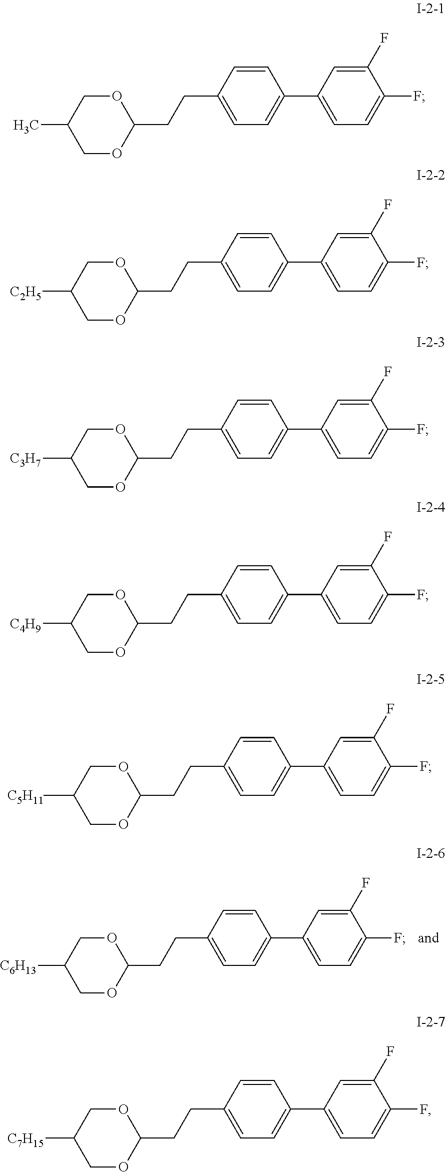

the compound of general formula I-3 is selected from a group consisting of the following compounds:

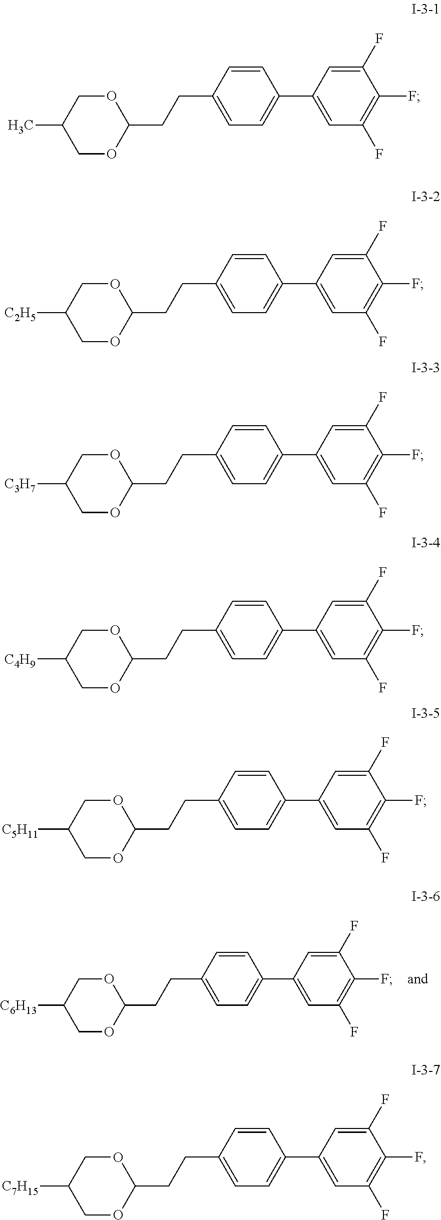

the compound of general formula I-4 is selected from a group consisting of the following compounds:

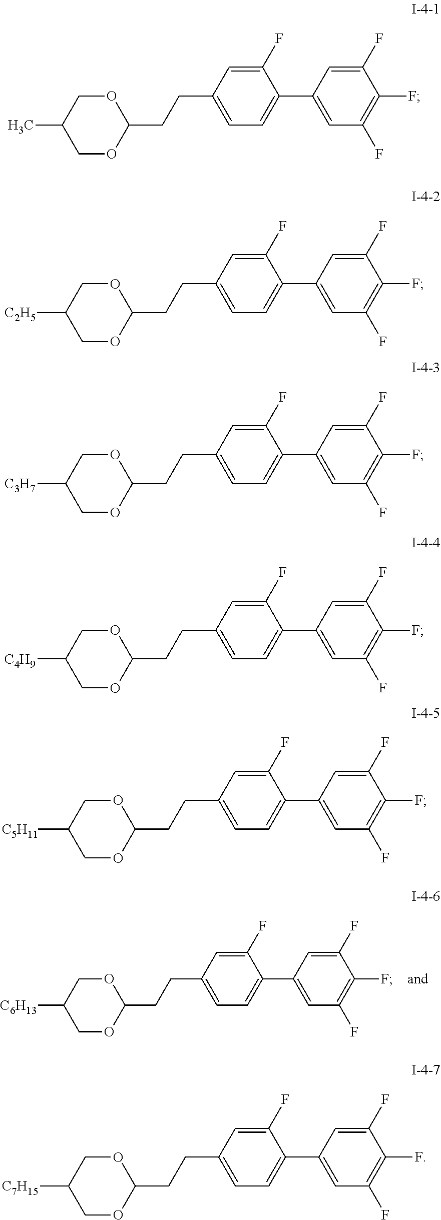

It should be noted that, simple modifications to chain length of the above compounds also fall into the protection scope of the compounds of the present invention.

The compound of general formula I of the present invention can enable the liquid crystal composition comprising the same to have larger dielectric anisotropy, thereby reducing the driving voltage of liquid crystal display devices; the compound of general formula I can make it easy to control the value of the optical anisotropy of the liquid crystal composition comprising the same to a desired level; the compound of general formula I can enable the liquid crystal composition comprising the same to have higher photo-thermal stability, and can be applicable to a harsh external environment; the compound of general formula I can enable the liquid crystal composition comprising the same to have good nematic phase stability where the compound of general formula I is less prone to crystallization when compatible with other liquid crystal compounds.

The compound of general formula I of the present invention is particularly suitable for use in a TFT-type liquid crystal composition as well as various other purposes. For example, there are liquid crystal compositions for use in TN-type, guest-host type, and polymer dispersion type liquid crystal display elements, dynamic dispersion type and STN-type, a ferroelectric liquid crystal composition, an anti-ferroelectric liquid crystal composition, and liquid crystal compositions for use in in-plane switching type, OCB-type and R—OCB-type.

In another aspect, the present invention further provides a liquid crystal composition comprising at least one compound of general formula I above.

In some embodiments of the present invention, preferably, the liquid crystal composition comprises at least one compound of general formula I-3 which provides 1-50% of the total weight of the liquid crystal composition.

In some embodiments of the present invention, preferably, the liquid crystal composition further comprises at least one compound selected from a group consisting of the compounds of general formulas II-1-II-4:

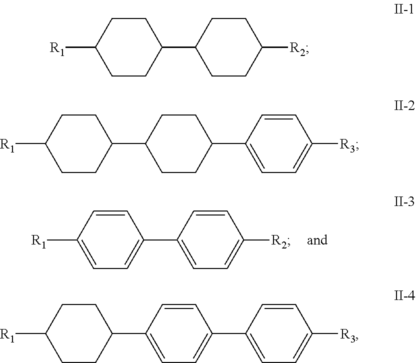

in which,

R₁ and R₂ each independently represents a C₁₋₁₂ linear or branched alkyl, or a C₂₋₁₂ linear or branched alkenyl;

R₃ represents a C₁₋₁₂ linear or branched alkyl or alkoxy, or a C₂₋₁₂ linear or branched alkenyl or alkenoxy.

In some embodiments of the present invention, further preferably, R₁ and R₂ each independently represents a C₁₋₇ chain alkyl, or a C₂₋₇ chain alkenyl; and R₃ represents a C₁₋₇ chain alkyl or chain alkoxy, or a C₂₋₇ chain alkenyl or chain alkenoxy.

In some embodiments of the present invention, the liquid crystal composition preferably comprises at least one compound in which at least one of R₁, R₂ and R₃ represents a C₂₋₇ chain alkenyl.

In some embodiments of the present invention, the liquid crystal composition preferably comprises at least one compound of general formula II-1 which provides 10-85% of the total weight of the liquid crystal composition.

In some embodiments of the present invention, the compound of general formula II-1 further preferably provides 15-55% of the total weight of the liquid crystal composition.

In some embodiments of the present invention, the compound of general formula II-1 still further preferably provides 25-50% of the total weight of the liquid crystal composition.

In some embodiments of the present invention, the compound of general formula I-3 further preferably provides 2-25% of the total weight of the liquid crystal composition.

In some embodiments of the present invention, the liquid crystal composition further preferably comprises at least one compound selected from general formula II-1-a and general formula II-1-b:

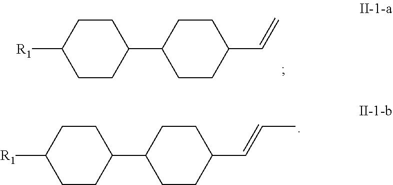

In still another aspect, the present invention further provides a photoelectric display device comprising the liquid crystal composition above.

### Beneficial Effects

The compound of general formula I provided by the present invention enables the liquid crystal composition comprising the same to maintain larger dielectric anisotropy, have a desired level of optical anisotropy, higher photo-thermal stability and nematic phase stability, and have a wide range of applicabilities, especially applicable to the IPS-type and TN-TFT-type liquid crystal display devices.

### DETAILED EMBODIMENTS

The present invention will be illustrated by combining the detailed embodiments below. It should be noted that, the following Examples are exemplary embodiments of the present invention, which are only used to illustrate the present invention, not to limit it. Other combinations and various modifications within the conception of the present invention are possible without departing from the subject matter and scope of the present invention.

For the convenience of the expression, the group structures of the liquid crystal compositions in the following Examples are represented by the codes listed in Table 1:

TABLE 1
Codes of the group structures of liquid crystal compounds

| Unit structure of group | Code | Name of group |
|---|---|---|
| 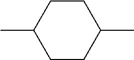 | C | 1,4-cyclohexylidene |
| 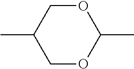 | D | 1,3-dioxane-2,5-diyl |
| 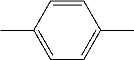 | P | 1,4-phenylene |
| 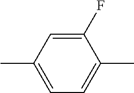 | G | 2-fluoro-1,4-phenylene |
| 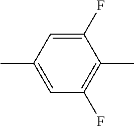 | U | 2,5-difluoro-1, 4-phenylene |
| 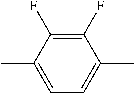 | W | 2,3-difluoro-1, 4-phenylene |
| —CH₂CH₂— | 2 | ethyl bridge bond |
| —OCF₃ | OCF3 | trifluoromethoxy |
| —F | F | fluorine substituent |
| —O— | O | oxygen substituent |
| —CF₂O— | 1(2F)O or Q | difluoro ether group |
| —CH₂O— | 1O | methyleneoxy |
| —COO— | E | ester bridge bond |
| —CₙH₂ₙ₊₁ or | n or m (n and m each | alkyl |
| —CₘH₂ₘ₊₁ | represents a positive | |
| | integer of 1-12) | |
| —CH═CH— or | V | ethenyl |
| —CH═CH₂ | | |

Taking a compound with the following structural formula as an example:

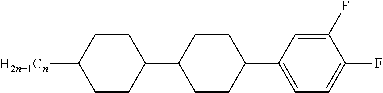

Represented by the codes listed in Table 1, this structural formula can be expressed as nCCGF, in which, n in the code represents the number of the carbon atoms of the alkyl on the left, for example, n is “3”, meaning that the alkyl is —C₃H₇; C in the code represents cyclohexyl, G represents 2-fluoro-1,4-phenylene, and F represents fluoro.

The abbreviated codes of the test items in the following Examples are as follows:
Cp (° C.) clearing point (nematic-isotropy phases transition temperature)
Δn optical anisotropy (589 nm, 25° C.)
Δε dielectric anisotropy (1 KHz, 25° C.)
VHR (UV) voltage holding ratio after UV-irradiation for 20 min (%)
VHR (high temperature) voltage holding ratio after degradation at 150° C. for 1 h (%)

In which,

the optical anisotropy is tested using an Abbe Refractometer under a sodium lamp (589 nm) light source at 25° C.;

Δε=ε_{|}-ε_{⊥}, in which, ε_{|} is a dielectric constant parallel to the molecular axis, ε_{⊥} is a dielectric constant perpendicular to the molecular axis, with the test conditions: 25° C., 1 KHz, TN90-type test cell with a cell gap of 7 m.

VHR (UV) is tested using a TOY06254 liquid crystal physical property evaluation system; the test temperature is 60° C., the test voltage is 5 V, the test time is 166.7 ms, and the UV-irradiation time is 20 min;

VHR (high temperature) is obtained by testing a liquid crystal composition, which is degraded at 150° C. for 1 h, using a TOY06254 type liquid crystal physical property evaluation system; the test temperature is 60° C., the test voltage is 5 V, and the test time is 166.7 ms.

The compound of general formula I of the present invention may be prepared by conventional organic synthesis methods. The methods for introducing a target terminal group, a cyclic group, and a linking group into a starting material can be found in the following literatures: Organic Synthesis (John Wiley & Sons Inc.), Organic Reactions (John Wiley & Sons Inc.), Comprehensive Organic Synthesis (Pergamon Press), and the like.

The methods for generating a linking group in the compound of general formula I can refer to the following schemes, wherein MSG¹ or MSG² is a monovalent organic group having at least one ring, and a plurality of MSG¹ (or MSG²) used in the following schemes can be the same or different.

### (1) Synthesis of Single Bond

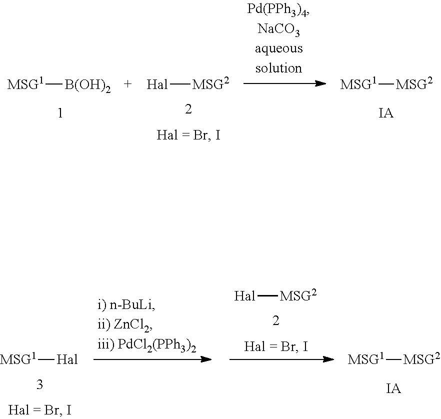

Compound (1A) with a single bond is prepared by allowing an aryl boronic acid (21) to react, in the presence of an aqueous carbonate solution and a catalyst such as tetrakis(triphenylphosphine)palladium, with compound (2) prepared according to a well-known method. The compound (1A) may also prepared by allowing compound (3) prepared according to a well-known method to react with n-butyllithium and subsequently with zinc chloride, and further with compound (2) in the presence of a catalyst such as dichlorobis(triphenylphosphine)palladium.

### (2) Synthesis of —CH₂CH₂—

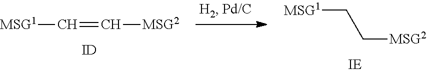

Compound (1E) may be prepared by hydrogenating compound (1D) with a catalyst such as palladium on carbon.

For rings such as 1,4-cyclohexylene, 1,3-dioxane-2,5-diyl, 1,4-phenylene, 2-fluoro-1,4-phenylene, 2,3-difluoro-1,4-phenylene, 2,5-difluoro-1,4-phenylene, 2,6-difluoro-1,4-phenylene, 2,3,5,6-tetrafluoro-1,4-phenylene and the like, the starting materials are already commercially available or their synthesis methods are well-known.

The preferred synthetic methods of representative compounds are illustrated below.

### Example 1

### Synthesis of Compound I-3-4

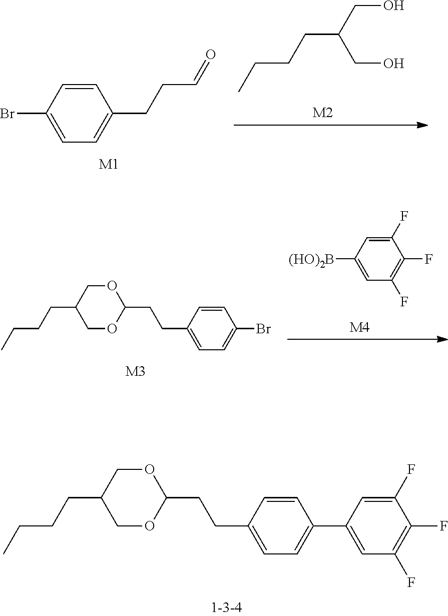

The specific preparation process is as follows:

(1) Synthesis of Compound M3

To a 500 mL three-necked flask are added 10.6 g compound M1, 6.6 g compound M2, 250 mL toluene, and 0.1 g p-toluenesulfonic acid, and the mixture is heated under reflux and watershed for 3 h. After the reaction is completed, it is post-treated and purified via column chromatography to obtain 13.7 g compound M3 (GC>97%, yield: 84%).

### (2) Synthesis of Compound I-3-4

To a 500 mL three-necked flask are added 3.3 g compound M3, 1.8 g compound M4, 100 mL toluene, 50 mL anhydrous ethanol, 50 mL water, and 4.2 g anhydrous sodium carbonate. 0.1 g of tetrakis (triphenylphosphine) palladium is added under the atmosphere of nitrogen and heated under reflux for 6 h. It is post-treated and purified via column chromatography and recrystallization to obtain 1.2 g compound I-3-4 as white solid (GC>99%, yield: 32%).

MS: 55(100%), 97(60%), 143(60%), 170(51%), 221(77%), 264(23%), 378(5%).

According to the synthetic method of compound I-3-4, other compounds that match general formula I above can be synthesized separately by the simple replacement of compounds M1 and/or M4 (not repeated here).

### Example 2

The liquid crystal composition of Example 2 is prepared according to each compound and weight percentage listed in Table 2 and then tested for performance by filling the same between two substrates of a liquid crystal display device. The test data is shown in the Table below:

TABLE 2
Formulation of the liquid crystal composition and its test performances

| | | | Test results for the | |
|---|---|---|---|---|
| Code of | Code of | Content | performance | |
| component | structure | percentage | parameters | |
| 3CCV | II-1-a | 30 | Cp | 61 |
| 5CCV | II-1-a | 10 | Δn | 0.08 |
| 3CC4 | II-1 | 10 | Δε | 2.7 |
| 5PP1 | II-3 | 6 | | |
| 5PP2 | II-3 | 6 | | |
| VCCP3 | II-2 | 15 | | |
| 3CPP2 | II-4 | 5 | | |
| 3D2PUF | I-3-3 | 6 | | |
| 4D2PUF | I-3-4 | 6 | | |
| 5D2PUF | I-3-5 | 6 | | |

| | |
|---|---|
| Total | 100 |

### Example 3

The liquid crystal composition of Example 3 is prepared according to each compound and weight percentage listed in Table 3 and then tested for performance by filling the same between two substrates of a liquid crystal display device. The test data is shown in the Table below:

TABLE 3
Formulation of the liquid crystal composition and its test performances

| | | | Test results for the | |
|---|---|---|---|---|
| Code of | Code of | Content | performance | |
| component | structure | percentage | parameters | |
| 3CCV | II-1-a | 27 | Cp | 68 |
| 5CCV | II-1-a | 8 | Δn | 0.088 |
| 3CC4 | II-1 | 5 | Δε | 2.3 |
| 5PP1 | II-3 | 9 | | |
| 5PP2 | II-3 | 9 | | |
| VCCP3 | II-2 | 15 | | |
| V2CCP2 | II-2 | 12 | | |
| 3D2PUF | I-3-3 | 5 | | |
| 4D2PUF | I-3-4 | 5 | | |
| 5D2PUF | I-3-5 | 5 | | |

| | |
|---|---|
| Total | 100 |

### Example 4

The liquid crystal composition of Example 4 is prepared according to each compound and weight percentage listed in Table 4 and then tested for performance by filling the same between two substrates of a liquid crystal display device. The test data is shown in the Table below:

TABLE 4
Formulation of the liquid crystal composition and its test performances

| | | | Test results for the | |
|---|---|---|---|---|
| Code of | Code of | Content | performance | |
| component | structure | percentage | parameters | |
| 3CCV | II-1-a | 25 | Cp | 77 |
| 3CCV1 | II-1-b | 3 | Δn | 0.09 |
| 5CCV | II-1-a | 4 | Δε | 1.7 |
| 5CC2 | II-1 | 3 | | |
| 5PP1 | II-3 | 9 | | |
| 5PP2 | II-3 | 9 | | |
| VCCP3 | II-2 | 13 | | |
| 3CPP2 | II-4 | 5 | | |
| VCCP2 | II-2 | 10 | | |
| VCCP4 | II-2 | 8 | | |
| 3D2PUF | I-3-3 | 3 | | |
| 4D2PUF | I-3-4 | 5 | | |
| 5D2PUF | I-3-5 | 3 | | |

| | |
|---|---|
| Total | 100 |

### Example 5

The liquid crystal composition of Example 5 is prepared according to each compound and weight percentage listed in Table 4 and then tested for performance by filling the same between two substrates of a liquid crystal display device. The test data is shown in the Table below:

TABLE 5
Formulation of the liquid crystal composition and its test performances

| | | | Test results for the | |
|---|---|---|---|---|
| Code of | Code of | Content | performance | |
| component | structure | percentage | parameters | |
| 3CCV | II-1-a | 25 | Cp | 82 |
| 5CCV | II-1-a | 5 | Δn | 0.091 |
| 3PP4 | II-3 | 6 | Δε | 1.2 |
| 5PP1 | II-3 | 8 | | |
| 5PP2 | II-3 | 8 | | |
| VCCP1 | II-2 | 10 | | |
| VCCP2 | II-2 | 10 | | |
| VCCP3 | II-2 | 10 | | |
| VCCP4 | II-2 | 10 | | |
| 4D2PUF | I-3-4 | 8 | | |

| | |
|---|---|
| Total | 100 |

### Example 6

The liquid crystal composition of Example 6 is prepared according to each compound and weight percentage listed in Table 6 and then tested for performance by filling the same between two substrates of a liquid crystal display device. The test data is shown in the Table below:

TABLE 6
Formulation of the liquid crystal composition and its test performances

| | | | Test results for the | |
|---|---|---|---|---|
| Code of | Code of | Content | performance | |
| component | structure | percentage | parameters | |
| 2CCV | II-1-a | 10 | Cp | 45 |
| 3CCV | II-1-a | 50 | Δn | 0.05 |
| 4CCV | II-1-a | 10 | Δε | 2 |
| 5CCV | II-1-a | 5 | | |
| VCC2V | II-1-a | 5 | | |
| VCCV | II-1-a | 5 | | |
| 3D2PUF | I-3-3 | 5 | | |
| 4D2PUF | I-3-4 | 5 | | |
| 5D2PUF | I-3-5 | 5 | | |

| | |
|---|---|
| Total | 100 |

### Application Example

The compounds of general formula I in Example 2, Example 5 and Example 6 above are replaced with compounds DB1, DB2 and DB3 respectively, to obtain Comparative Example 1, Comparative Example 2, and Comparative Example 3. The dielectric anisotropy, VHR (UV), VHR (high temperature) and nematic phase stability of the liquid crystal compositions of Comparative Examples 1-3 are tested, and the results are shown in Table 7 below:

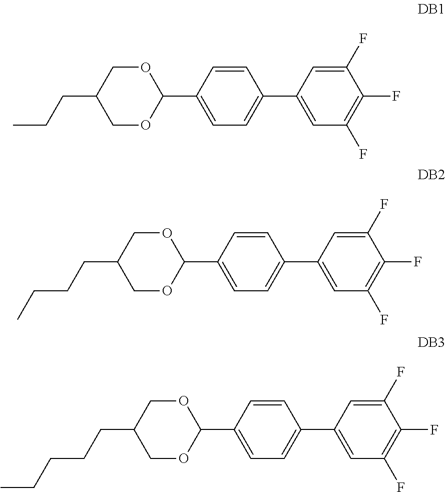 [SMILES: CCCC1COC(c2ccc(-c3cc(F)c(F)c(F)c3)cc2)OC1 ; CCCCC1COC(c2ccc(-c3cc(F)c(F)c(F)c3)cc2)OC1 ; CCCCCC1COC(c2ccc(-c3cc(F)c(F)c(F)c3)cc2)OC1]

TABLE 7

| | | | VHR (high | |
|---|---|---|---|---|
| | Δε | VHR (UV) | temperature) | nematic phase stability |
| Example 2 | 2.7 | 96.5% | 97% | The nematic phase is held |
| | | | | for greater than 24 h |
| Example 5 | 1.2 | 96.7% | 97% | The nematic phase is held |
| | | | | for greater than 24 h |
| Example 6 | 2 | 96.8% | 97% | The nematic phase is held |
| | | | | for greater than 24 h |
| Comparative | 2.8 | 95.8% | 97% | The nematic phase is held |
| Example 1 | | | | for less than 1 h |
| Comparative | 1.2 | 96.2% | 97% | The nematic phase is held |
| Example 2 | | | | for less than 1 h |
| Comparative | | | | The nematic phase is held |
| Example 3 | 2 | 96.2% | 97% | for less than 1 h |

Note:
Nematic phase stability refers to whether the liquid crystal composition is in a nematic phase and the hold time of the nematic phase at room temperature.

As can be seen from Table 7 above, the liquid crystal compositions comprising the compound of general formula I of the present invention maintain larger dielectric anisotropy, have better photothermal stability and nematic phase stability, and are applicable to IPS-type and TN-TFT-type liquid crystal display devices, relative to the liquid crystal compositions comprising the compounds DB1, DB2 and DB3.

The Examples illustrated above are merely preferred embodiments of the present invention, and are not intended to limit the present invention in any form. Although the present invention has been disclosed as above with preferred embodiments, it is not intended to limit the present invention. Without departing from the scope of the technical solutions of the present invention, any person skilled in the art should be able to use the disclosed technical contents to make some changes or modifications to obtain equivalent embodiments with equivalent changes. Any simple alterations, equivalent changes and modifications made to the above embodiments according to the technical essence of the present invention without departing from the content of the technical solution of the present invention still fall into the scope of the technical solutions of the present invention.

### INDUSTRIAL APPLICABILITY

The liquid crystal compound and liquid crystal composition and photoelectric display device thereof related in the present invention can be applied to the field of liquid crystal.

1. A compound having the structure of general formula I: 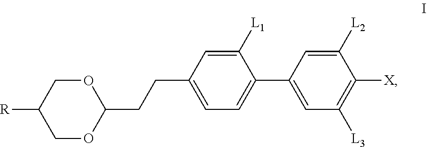
in which,
R represents a C₁₋₁₂ linear or branched alkyl or alkoxy, a C₂₋₁₂ linear or branched alkenyl or alkenoxy, or a C₃₋₁₂ cycloalkyl, wherein one or more —CH₂— in the alkyl or alkoxy, alkenyl or alkenoxy, or cycloalkyl can be replaced by —O—, provided that oxygen atoms are not directly connected;
L₁, L₂ and L₃ each independently represents —H or —F; and
X represent —F, a C₁₋₁₂ linear or branched fluorinated alkyl or fluorinated alkoxy, or a C₂₋₁₂ linear or branched fluorinated alkenyl or fluorinated alkenoxy.

2. The compound according to claim 1, wherein X represents —F, —CF₃ or —OCF₃.

3. The compound according to claim 2, wherein the compound of general formula I is selected from a group consisting of the following compounds:

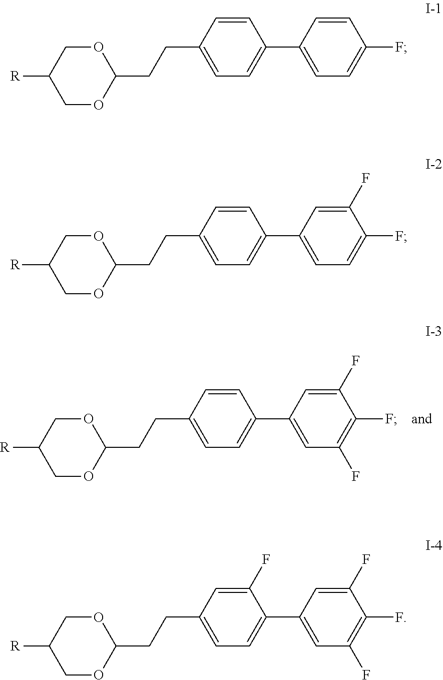

4. A liquid crystal composition, comprising at least one compound of general formula I according to claim 1.

5. The liquid crystal composition according to claim 4, wherein the liquid crystal composition comprises at least one compound of general formula I-3 which provides 1-50% of the total weight of the liquid crystal composition.

6. The liquid crystal composition according to claim 5, wherein the liquid crystal composition further comprises at least one compound selected from a group consisting of the compounds of general formulas II-1 through II-4: 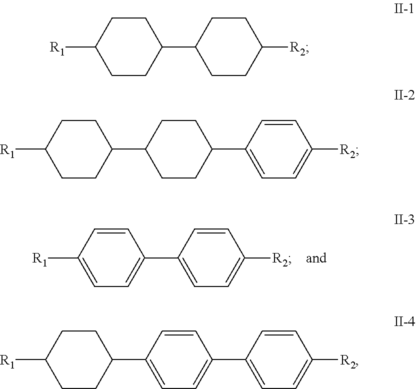
in which,
R₁ and R₂ each independently represents a C₁₋₁₂ linear or branched alkyl, or a C₂₋₁₂ linear or branched alkenyl; and
R₃ represents a C₁₋₁₂ linear or branched alkyl or alkoxy, or a C₂₋₁₂ linear or branched alkenyl or alkenoxy.

7. The liquid crystal composition according to claim 6, wherein the liquid crystal composition comprises at least one compound of general formula II-1 which provides 10-85% of the total weight of the liquid crystal composition.

8. The liquid crystal composition according to claim 7, wherein the compound of general formula II-1 provides 15-55% of the total weight of the liquid crystal composition.

9. The liquid crystal composition according to claim 8, wherein the compound of general formula II-1 provides 25-50% of the total weight of the liquid crystal composition.

10. The liquid crystal composition according to claim 5, wherein the compound of general formula I-3 provides 2-25% of the total weight of the liquid crystal composition.

11. A photoelectric display device comprising the liquid crystal composition according to claim 4.
